(19) United States
(12) Patent Application Publication
(10) Pub. No.: US 2024/0360057 A1
(43) Pub. Date: Oct. 31, 2024

(54) METHOD FOR EXTRACTING ORGANIC HALOGEN COMPOUND

(71) Applicant: MIURA CO., LTD., Matsuyama-shi, Ehime (JP)

(72) Inventors: Hiroyuki FUJITA, Matsuyama-shi (JP); Shota NONAKA, Matsuyama-shi (JP)

(73) Assignee: MIURA CO., LTD., Matsuyama-shi, Ehime (JP)

(21) Appl. No.: 18/026,066

(22) PCT Filed: Apr. 20, 2021
(86) PCT No.: PCT/JP2021/015951

§ 371 Date: Mar. 13, 2023

(30) Foreign Application Priority Data
Sep. 28, 2020 (JP) 2020-162753

(51) Int. Cl. C07C 17/389; B01D 15/18; B01D 15/20; B01D 15/22; B01J 20/10; B01J 20/20; B01J 20/28; B01J 20/283; G01N 30/02; G01N 30/72

(52) U.S. Cl. CPC C07C 17/389; B01D 15/1871; B01D 15/203; B01D 15/22; B01J 20/103; B01J 20/20; B01J 20/28016; B01J 20/283; G01N 30/7206; G01N 2030/025

(57) ABSTRACT

An extraction column (1) is used, which includes a first column (10) having an adsorbent layer (100) with a silver nitrate silica gel layer (110) and a sulfuric silica gel layer (130) and a second column (20) detachably coupled to the first column (10) and filled with a trapping layer (200) capable of trapping an organic halogen compound. The trapping layer (200) includes a trapping material having a granular carrier containing aluminum oxide and fine particle-shaped silver held on a surface of the carrier. An aliphatic hydrocarbon solvent is supplied to the adsorbent layer (100) to which an organic halogen compound-containing solution has been added, and passes through the adsorbent layer (100) and the trapping layer (200) in this order. A solvent for extraction of the organic halogen compound is supplied to the trapping layer (200) from the lower side of the second column (20), and the extraction solvent having passed through the trapping layer (200) is obtained through a branched path (22).

### TECHNICAL FIELD

The present application claims a priority based on Japanese Application No. 2020-162753 filed in Japan on Sep. 28, 2020, and the contents of which are incorporated herein by reference.

The present invention relates to a method for extracting an organic halogen compound, and specifically relates to a method for extracting an organic halogen compound from a solution containing the organic halogen compound and an impurity.

### BACKGROUND ART

For a bottom sediment, soil, incinerated ash caused from an incineration facility, food, a biological sample such as blood or breast milk, environmental water or industrial drainage water such as seawater, river water, lake water, or groundwater, atmospheric air, exhaust from an incineration facility and the like, evaluation of a contamination status due to an organic halogen compound about which toxicity to a biological body has been concerned has been demanded. According to, e.g., Act on Special Measures against Dioxins (Act No. 105 of 1999), for dioxins known as an environmental pollutant having strong toxicity to a biological body, environmental standards have been established, emission control standards for each specific facility have been established, and regular quantitative evaluation has been demanded. According to food control standards (COMMISSION REGULATION (EU) No 1259/2011) in European Union (EU), dioxins and predetermined polychlorinated biphenyls which do not fall into the category of dioxins have been specified as organic halogen compounds targeted for control for food such as meat including beef and pork, animal oil and fat, egg, and vegetable oil including olive oil, and control values have been set for these organic halogen compounds and quantitative evaluation has been also demanded for these organic halogen compounds. Further, according to Method 1668C, April 2010 established by the United States Environmental Protection Agency (EPA), quantitative evaluation of polychlorinated biphenyls in water, soil, a bottom sediment, a living organism, or a body tissue has been demanded, and an evaluation method therefor has been specified.

Normally, in evaluation of the contamination status due to the organic halogen compound, the organic halogen compound is, from a sample targeted for evaluation, extracted using a solvent such as an aliphatic hydrocarbon solvent such as hexane or an aromatic hydrocarbon solvent such as toluene, and a solution with the organic halogen compound obtained by such extraction is analyzed by a method using a high-sensitive analyzer such as a gas chromatograph/mass spectrometer (GC/MS) or a gas chromatograph/electron capture detector (GC/ECD).

Normally, when the organic halogen compound is extracted, for analysis, from the evaluation target sample, various organic compounds are simultaneously extracted as impurities together with the organic halogen compound, and for this reason, there are probabilities that the analyzer is contaminated with the impurities and the impurities influence an analysis result for the organic halogen compound if the extract is directly used as the analysis sample. Thus, the organic halogen compound extract from the evaluation target sample normally requires pretreatment for removing the impurities. However, in this pretreatment, the impurities need to be removed without a failure to recover the organic halogen compound targeted for analysis. For example, polychlorinated biphenyls (hereinafter referred to as PCBs) are a collective term of biphenyls that hydrogen atoms are substituted for chlorine atoms. Based on a substituted chlorine number, there are ten types of homologues from monochlorobiphenyl to decachlorobiphenyl. Moreover, based on the substituted chlorine number and a substituted chlorine position, there are 209 types of homologues. For this reason, in order to analyze, with a high accuracy, the PCBs extracted from the evaluation target sample, pretreatment in which the rate of recovery of each homologue of the PCBs is less likely to be lowered, i.e., high-accuracy pretreatment in which the impurities can be removed such that the rate of recovery remains within a public acceptable range, is demanded.

As one type of high-accuracy pretreatment, Non-Patent Literature 1 describes a pretreatment method for a hexane solution with PCBs extracted from a bottom sediment. In this pretreatment method, sulfuric acid treatment is repeated for the hexane solution which is a sample, and after such treatment, the hexane solution is rinsed with a saturated sodium chloride solution and is concentrated. Then, the concentrated hexane solution is further treated with a silica gel column including sodium sulfate, and in this manner, the PCBs are extracted using hexane from the silica gel column. This pretreatment method can effectively remove the impurities without lowering of the rate of recovery of each homologue of the PCBs, but requires a long time until completion due to a manual work in large part of a process and has a limitation on a treatable amount within a certain period of time.

### PRIOR ART LITERATURE

### Non-Patent Literature

Non-Patent Literature 1: Sediment Monitoring Methods (II. 6.4), Environmental Management Bureau, Ministry of the Environment, August 2012

### DISCLOSURE OF THE INVENTION

### Problems to be Solved by the Invention

The present invention is intended to extract, without a failure to recover an organic halogen compound, the organic halogen compound from a solution containing the organic halogen compound and an impurity by simple operation.

### Solution to the Problems

The present invention relates to a method for extracting an organic halogen compound from a solution containing the organic halogen compound and an impurity. The extraction method includes a step 1 of adding the solution to an adsorbent layer capable of treating the impurity, a step 2 of supplying an aliphatic hydrocarbon solvent to the adsorbent layer to which the solution has been added and causing the aliphatic hydrocarbon solvent to pass through the adsorbent layer, a step 3 of supplying the aliphatic hydrocarbon solvent having passed through the adsorbent layer to a trapping layer capable of trapping the organic halogen compound and causing the aliphatic hydrocarbon solvent to pass through the trapping layer, a step 4 of supplying a solvent for extraction of the organic halogen compound to the trapping layer through which the aliphatic hydrocarbon solvent has passed and causing the extraction solvent to pass through the trapping layer, and a step 5 of obtaining the extraction solvent having passed through the trapping layer. The trapping layer includes a trapping material having a carrier containing aluminum oxide and transition metal held on a surface of the carrier.

In the extraction method, the impurity in the solution added to the adsorbent layer in the step 1 is treated. When the aliphatic hydrocarbon solvent is supplied to the adsorbent layer in the step 2, the organic halogen compound in the solution is dissolved in the aliphatic hydrocarbon solvent, and passes through the adsorbent layer. When the aliphatic hydrocarbon solvent having passed through the adsorbent layer is supplied to the trapping layer and passes through the trapping layer in the step 3, the organic halogen compound in the aliphatic hydrocarbon solvent from the adsorbent layer is trapped by the trapping layer, and the aliphatic hydrocarbon solvent passes through the trapping layer with the organic halogen compound removed from the aliphatic hydrocarbon solvent. When the extraction solvent is supplied to the trapping layer in the step 4, the extraction solvent extracts the organic halogen compound trapped by the trapping layer while passing through the trapping layer. Thus, the extraction solvent obtained from the trapping layer in Step 5 turns into an extract containing the organic halogen compound contained in the solution.

The carrier of the trapping material used in the extraction method of the present invention is, for example, in the form of a grain. Moreover, the transition metal of the trapping material is held, for example, in the form of fine particles on the surface of the carrier. Further, the transition metal of the trapping material is, for example, at least one selected from a group consisting of silver, copper, and nickel.

One aspect of the extraction method of the present invention further includes a step 6 of supplying the aliphatic hydrocarbon solvent having passed through the adsorbent layer in the step 3 to a precedent trapping layer containing a carbon-based material or active magnesium silicate and causing the aliphatic hydrocarbon solvent to pass through the precedent trapping layer, supplying the aliphatic hydrocarbon solvent to the trapping layer and causing the aliphatic hydrocarbon solvent to pass through the trapping layer, and supplying the solvent for extraction of the organic halogen compound to the precedent trapping layer through which the aliphatic hydrocarbon solvent has passed and causing the extraction solvent to pass through the precedent trapping layer, and a step 7 of obtaining the extraction solvent having passed through the precedent trapping layer.

The solution to which the extraction method of the present invention is applicable is, for example, a solution containing an organic halogen compound extracted using a solvent from a collector having collected a material layer on a bottom in the hydrosphere or a land surface, food, a biological sample, environmental water, drainage water, electric insulating oil, incinerated ash, or a substance contained in gas.

Another aspect of the present invention relates to a column for extracting an organic halogen compound from a solution containing the organic halogen compound and an impurity. The extraction column includes a first column filled with an adsorbent layer capable of treating the impurity, and a second column detachably coupled to the first column and filled with a trapping layer capable of trapping the organic halogen compound. The trapping layer includes a trapping material having a carrier containing aluminum oxide and transition metal held on a surface of the carrier.

In one aspect of the column for extracting the organic halogen compound according to the present invention, the second column is further filled with a precedent trapping layer arranged between the adsorbent layer and the trapping layer and containing a carbon-based material or active magnesium silicate.

Yet another aspect of the present invention relates to a column for trapping an organic halogen compound contained in an aliphatic hydrocarbon solvent. The trapping column is filled with a trapping layer including a trapping material having a carrier containing aluminum oxide and transition metal held on a surface of the carrier.

### Effects of the Invention

The method for extracting the organic halogen compound according to the present invention includes the above-described steps 1 to 5, and therefore, the organic halogen compound can be extracted, without a failure to recover the organic halogen compound, from the solution containing the organic halogen compound and the impurity by simple operation.

The column for extracting the organic halogen compound according to the present invention and the column for trapping the organic halogen compound according to the present invention can be used in the method for extracting the organic halogen compound according to the present invention.

### BRIEF DESCRIPTION OF THE DRAWINGS

FIG. 1 is a schematic view of one form of an extraction column available for a method for extracting an organic halogen compound according to the present invention.

FIG. 2 is a schematic view of another form of the extraction column available for the method for extracting the organic halogen compound according to the present invention.

FIG. 3 is a view showing a variation of a first column used for the extraction column of each form.

FIG. 4 is a view showing another variation of the first column used for the extraction column of each form.

FIG. 5 is a view showing yet another variation of the first column used for the extraction column of each form.

### DESCRIPTION OF PREFERRED EMBODIMENTS

A method for extracting an organic halogen compound according to the present invention relates to a method for extracting an organic halogen compound from a solution containing the organic halogen compound.

The organic halogen compound targeted for extraction includes, for example, dioxins (polychlorinated dibenzo-p-dioxins (PCDDs) and polychlorinated dibenzofurans (PCDFs)), and polyhalogenated biphenyls and polybrominated diphenyl ethers such as polychlorinated biphenyls and polybrominated biphenyls. The solution containing the organic halogen compound and targeted for the extraction method of the present invention, i.e., an organic halogen compound-containing solution, is normally a solution containing the organic halogen compound extracted using a solvent from a target for which evaluation of, e.g., a contamination status due to the organic halogen compound is required, the target including, e.g., a collector such as a filter having collected material layers on a bottom in the hydrosphere and a land surface, such as a bottom sediment and soil; food such as an agricultural crop, meat, and seafood; body fluid such as breast milk and blood; a biological sample such as an organ and a tissue; environmental water such as river water, lake water, and groundwater; drainage water such as industrial wastewater and domestic wastewater; electric insulating oil; incinerated ash caused from an incineration facility; and a substance contained in gas such as environmental air or exhaust from an incineration facility. The extraction solvent for obtaining such a solution is not specifically limited as long as the organic halogen compound can be dissolved in the solvent, and is normally an organic solvent. The organic solvent to be used includes, for example, an aliphatic hydrocarbon solvent, specifically a non-polar aliphatic hydrocarbon solvent with a carbon number of 5 to 10, such as n-hexane, iso-octane, nonane, or decane; an aromatic hydrocarbon solvent such as toluene or xylene: or a polar organic solvent such as acetone, diethyl ether, or dichloromethane. Note that for an extract obtained in such a manner that the organic halogen compound is extracted using the aromatic hydrocarbon solvent, the solvent is preferably substituted for the above-described aliphatic hydrocarbon solvent in application to the extraction method of the present invention.

The organic halogen compound-containing solution contains, together with the organic halogen compound, various impurities derived from the above-described evaluation target for which evaluation of, e.g., the contamination status due to the organic halogen compound is required, mainly various organic substances other than the organic halogen compound. The impurities include, e.g., an aromatic compound such as polycyclic aromatic hydrocarbons and aliphatic hydrocarbons such as paraffins.

### First Embodiment

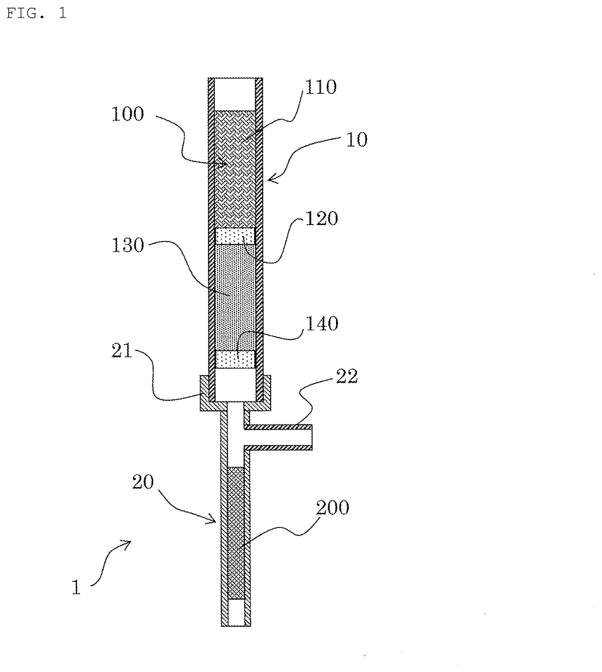

An exemplary embodiment (a first embodiment) of an extraction column used for performing the method for extracting the organic halogen compound according to the present invention will be described with reference to FIG. 1. In the figure, an extraction column 1 mainly includes a first column 10 and a second column 20 (one form of a column for trapping the organic halogen compound according to the present invention) coupled to the first column 10 to form a series of flow path system, and is installed in a standing state.

The first column 10 is a cylindrical member opening at both ends, and is made of a material having at least solvent resistance, chemical resistance, and thermal resistance, such as glass, resin, or metal having these properties. The first column 10 has, at an outer peripheral surface of a lower end portion as viewed in the figure, a thread portion (not shown) to be coupled to the second column 20, and is filled with an adsorbent layer 100. The adsorbent layer 100 is for treating the impurity contained in the organic halogen compound-containing solution, and for example, is for decomposing the impurity or trapping the impurity or a decomposition product thereof. The adsorbent layer 100 is a multilayer silica gel layer in which a silver nitrate silica gel layer 110, a first active silica gel layer 120, a sulfuric silica gel layer 130, and a second active silica gel layer 140 are arranged in this order from above in the first column 10.

The silver nitrate silica gel layer 110 is a layer made of silver nitrate silica gel. The silver nitrate silica gel used herein is prepared in such a manner that after a silver nitrate aqueous solution has been uniformly added to a surface of silica gel (normally, active silica gel of which the degree of activity has been enhanced by heating) in the form of a grain with a particle size of about 40 to 210 μm, moisture is removed by heating under a reduced pressure. The amount of silver nitrate supported on the silica gel is normally preferably set to 5 to 20% of the mass of the silica gel. In a case where the support amount is less than 5%, there is a probability that an impurity treatment effect is degraded in the silver nitrate silica gel layer 110. Conversely, in a case where the support amount exceeds 20%, the organic halogen compound is easily trapped because of a great silver ion amount in the silver nitrate silica gel layer 110, leading to a probability that part of the organic halogen compound is less likely to be recovered in extraction of the organic halogen compound.

The moisture content of the silver nitrate silica gel layer 110 is generally preferably set to 2 to 10% of the mass of the silica gel and more preferably 3.5 to 5%. In a case where the moisture content is 2% or less, the organic halogen compound is easily trapped because of a silver ion activity being enhanced in the silver nitrate silica gel layer 110, leading to a probability that part of the organic halogen compound is less likely to be recovered in extraction of the organic halogen compound. Conversely, in a case where the moisture content exceeds 10%, there is a probability that the impurity treatment effect is degraded in the silver nitrate silica gel layer 110.

The density of the filled silver nitrate silica gel in the silver nitrate silica gel layer 110 is not specifically limited, but is normally preferably set to 0.3 to 0.8 g/cm³ and more preferably 0.4 to 0.7 g/cm³. In a case where the density is less than 0.3 g/cm³, there is a probability that an impurity treatment efficiency is degraded. Conversely, in a case where the density exceeds 0.8 g/cm³, the later-described aliphatic hydrocarbon solvent is less likely to pass through the adsorbent layer 100.

The sulfuric silica gel layer 130 is a layer made of sulfuric silica gel. The sulfuric silica gel used herein is prepared in such a manner that concentrated sulfuric acid is uniformly added to a surface of silica gel (normally, active silica gel of which the degree of activity has been enhanced by heating) in the form of a grain with a particle size of about 40 to 210 μm. The amount of concentrated sulfuric acid added to the silica gel is normally preferably set to 10 to 60% of the mass of the silica gel.

The density of the filled sulfuric silica gel in the sulfuric silica gel layer 130 is not specifically limited, but is normally preferably set to 0.3 to 1.1 g/cm³ and more preferably 0.5 to 1.0 g/cm³. In a case where the density is less than 0.3 g/cm³, there is a probability that the impurity treatment efficiency is degraded. Conversely, in a case where the density exceeds 1.1 g/cm³, the later-described aliphatic hydrocarbon solvent is less likely to pass through the adsorbent layer 100.

The first active silica gel layer 120 is arranged to avoid chemical reaction between the silver nitrate silica gel layer 110 and the sulfuric silica gel layer 130 due to direct contact therebetween, and is made of silica gel in the form of a grain with a particle size of about 40 to 210 μm. The silica gel used herein may be one of which the degree of activity has been enhanced as necessary by heating.

The second active silica gel layer 140 is made of silica gel similar to that of the first active silica gel layer 120, and is provided for adsorbing the decomposition product caused due to treatment of the impurity in the silver nitrate silica gel layer 110 and the sulfuric silica gel layer 130 and sulfuric acid eluted from the sulfuric silica gel layer 130 to prevent these components from moving to the second column 20.

In the adsorbent layer 100, a ratio between the silver nitrate silica gel layer 110 and the sulfuric silica gel layer 130 is set such that the mass ratio of the sulfuric silica gel layer 130 to the silver nitrate silica gel layer 110 is preferably 1.0 to 50 and more preferably 3.0 to 30. When the mass ratio of the sulfuric silica gel layer 130 exceeds 50, the percentage of the silver nitrate silica gel layer 110 is relatively low, and for this reason, there is a probability that the capacity of the adsorbent layer 100 for treating the impurity contained in the organic halogen compound-containing solution, specifically a capacity of adsorbing the impurity, is insufficient. Conversely, the mass ratio of the sulfuric silica gel layer 130 is less than 1.0, there is a probability that the capacity of the adsorbent layer 100 for treating the impurity contained in the organic halogen compound-containing solution, specifically a capacity of decomposing the impurity, is insufficient.

The second column 20 is basically a cylindrical member opening at both ends, and is made of a material similar to that of the first column 10. On the upper end side of the second column 20 as viewed in the figure, an attachment portion 21 into which the lower end portion of the first column 10 as viewed in the figure is insertable is formed. A thread portion (not shown) is formed at an inner peripheral surface of the attachment portion 21. The second column 20 further has a branched path 22 opening at a tip end below the attachment portion 21.

The second column 20 is, below the branched path 22, filled with a trapping layer 200. The trapping layer 200 includes a trapping material capable of trapping the organic halogen compound. The trapping material includes a carrier containing aluminum oxide and transition metal held on a surface of the carrier.

Aluminum oxide contained in the carrier is normally preferably intermediate alumina such as γ-alumina, δ-alumina, or θ-alumina, or may be any of basic alumina, neutral alumina, and acidic alumina. The degree of activity of aluminum oxide is not specifically limited. The carrier may be in any form as long as liquid permeability in the trapping layer 200 can be ensured, and may be in the form of, e.g., grain, porous pellet, or fiber and preferably the form of a grain with a particle size of 10 to 300 μm.

The type of transition metal held on the surface of the carrier is not specifically limited. The transition metal normally includes an element in Groups 3 to 11 in the periodic table, i.e., scandium, titanium, vanadium, chromium, manganese, iron, cobalt, nickel, copper, yttrium, zirconium, niobium, molybdenum, technetium, ruthenium, rhodium, palladium, silver, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, and gold, and also further includes zinc, cadmium, and mercury in Group 12. Two or more types of transition metal may be used in combination. Silver, copper, nickel, or any combination thereof is preferred as the transition metal because of a high capacity of trapping the organic halogen compound.

For example, the transition metal is held on the surface of the carrier with entirely or partially covering the surface of the carrier or with scattered in the form of fine particles on the surface of the carrier. In the latter case, the transition metal is particularly preferably held in the form of fine particles with a size of about μm to nm on the surface of the carrier.

The trapping material holding the transition metal on the surface of the carrier can be manufactured by the following steps, for example. First, a transition metal nitrate aqueous solution is prepared, and aluminum oxide is dipped in the aqueous solution. A dipping time is normally preferably about 5 to 30 minutes. Next, a hydroxide salt aqueous solution is added to the aqueous solution in which aluminum oxide is dipped, and transition metal oxide is generated on a surface of aluminum oxide. The oxide is generated through hydroxylation of the transition metal. Hydroxide salt used in this step is, for example, alkali metal hydroxide such as sodium or potassium or alkaline-earth metal hydroxide such as magnesium or calcium. Next, the aqueous solution is separated into solid and liquid. The collected solid is heated and dried in an oven, and thereafter, is fired in a tubular firing furnace under inert gas atmosphere of, e.g., nitrogen or reducing atmosphere of hydrogen gas. In this firing process, the transition metal oxide releases oxygen, and simple transition metal in the form of a grain is held on the surface of aluminum oxide which is the carrier. A temperature in drying of the solid separated from the aqueous solution is normally preferably set to 120 to 300° C. A drying time varies according to the temperature in drying, but is normally about 4 to 16 hours. A temperature in firing of the dried solid is normally preferably set to 500 to 800° C., and a firing time is preferably set to 0.5 to 4 hours. In this manufacturing method, nitrate ions in the aqueous solution are separated from the solid in separation into the solid and the liquid, and therefore, occurrence of nitrogen oxide (NOX) can be suppressed in firing of the solid and the trapping material can be safely mass-produced.

The method for manufacturing the trapping material may include not only the above-described method through oxidation of the transition metal, but also a reduction method. In this case, a reductant such as ascorbic acid or glucose is added to the above-described aqueous solution in which aluminum oxide is dipped, and by reduction of the transition metal in the form of nitrate, simple transition metal is deposited on aluminum oxide. Also, in this case, the aqueous solution to which the reductant has been added is separated into solid and liquid. The collected solid is heated and dried in the oven, and thereafter, is fired in the tubular firing furnace under inert gas atmosphere of, e.g., nitrogen. The conditions for drying and firing the solid can be set in a manner similar to that in the case of the method through oxidation of the transition metal.

In the above-described manufacturing method, the concentration of nitrate in the transition metal nitrate aqueous solution is not specifically limited. Note that the amount of aqueous solution mixed with aluminum oxide is normally preferably set such that the amount of transition metal with respect to the mass (g) of aluminum oxide is 0.5 to 20% and more preferably 2 to 10%.

The density of the filled trapping layer 200 is normally preferably set to 0.5 to 1.0 g/cm³ and more preferably 0.7 to 0.9 g/cm³. In a case where the filling density is less than 0.5 g/cm³, there is a probability that it is difficult to recover the organic halogen compound contained in the organic halogen compound-containing solution without a failure to recover the organic halogen compound. Conversely, in a case where the filling density exceeds 1.0 g/cm³, there is a probability that recovery of the organic halogen compound is insufficient or a pressure loss is great when the organic halogen compound is extracted using the later-described extraction solvent from the trapping layer 200.

The first column 10 is detachably coupled to the second column 20 in a liquid-tight manner in such a manner that the thread portion provided at the outer periphery of the lower end of the first column 10 is attached to the thread portion provided at the inner peripheral surface of the attachment portion 21 of the second column 20.

The size of the extraction column 1 can be set as necessary according to the amount of organic halogen compound-containing solution to be treated. For example, in a case where the amount of organic halogen compound-containing solution is about 1 to 20 mL, the first column 10 is preferably set such that the inner diameter of a portion, which can be filled with the adsorbent layer 100, of the first column 10 is 10 to 20 mm and the length thereof is about 100 to 300 mm, and the second column 20 is preferably set such that the inner diameter of the second column 20 is 3 to 10 mm and the length of a portion, which can be filled with the trapping layer 200, of the second column 20 is about 20 to 50 mm.

Next, the method for extracting the organic halogen compound from the organic halogen compound-containing solution by means of the above-described extraction column 1 will be described. In this extraction method, the extraction column 1 is installed in the standing state as shown in FIG. 1, and from the upper end opening, the organic halogen compound-containing solution is added onto the adsorbent layer 100 in the first column 10 (step 1). At this point, part of the adsorbent layer 100, i.e., the entirety of the silver nitrate silica gel layer 110 and the first active silica gel layer 120 and an upper portion of the sulfuric silica gel layer 130 are preferably heated.

The added organic halogen compound-containing solution penetrates an upper portion of the silver nitrate silica gel layer 110, and is heated together with the part of the adsorbent layer 100. The temperature of heating of the adsorbent layer is set to 35° C. or more, preferably 50° C. or more, and more preferably 60° C. or more. By such heating, part of the impurity other than the organic halogen compound contained in the organic halogen compound-containing solution reacts with the adsorbent layer 100, and is decomposed. In a case where the heating temperature is less than 35° C., the reaction between the impurity and the adsorbent layer 100 is less likely to progress, leading to a probability that part of the impurity easily remains in a later-described organic halogen compound extract. The upper limit of the heating temperature is not specifically limited, but considering safety, is normally preferably a boiling temperature or less.

In heating, the silver nitrate silica gel layer 110 and the sulfuric silica gel layer 130 are stacked with the first active silica gel layer 120 interposed therebetween, and therefore, reaction therebetween is suppressed.

Next, for a predetermined time from the start of heating, such as after a lapse of 10 to 60 minutes, the aliphatic hydrocarbon solvent is supplied to the adsorbent layer 100 in the first column 10 through the upper end opening, and then, passes through the adsorbent layer 100 (step 2). At this point, heating of the adsorbent layer 100 may be continued or stopped. The aliphatic hydrocarbon solvent supplied at this point is an aliphatic saturated hydrocarbon solvent which can dissolve the organic halogen compound and preferably has a carbon number of 5 to 8. For example, n-pentane, n-hexane, n-heptane, n-octane, iso-octane, or cyclohexane is preferably used. These solvents may be mixed upon use, as necessary.

The aliphatic hydrocarbon solvent supplied to the adsorbent layer 100 dissolves the organic halogen compound contained in the organic halogen compound-containing solution having penetrated the adsorbent layer 100, the decomposition product of the impurity, and the undecomposed remaining impurity, and passes through the adsorbent layer 100. At this point, part of the decomposition product and the impurity adsorbs to the silver nitrate silica gel layer 110, the first active silica gel layer 120, the sulfuric silica gel layer 130, and the second active silica gel layer 140. Moreover, the aliphatic hydrocarbon solvent passing through the adsorbent layer 100 is naturally cooled while passing through an unheated portion, i.e., a lower portion of the sulfuric silica gel layer 130 and the second active silica gel layer 140.

The aliphatic hydrocarbon solvent having passed through the adsorbent layer 100 flows into the second column 20 from the first column 10, and passes through the trapping layer 200. Then, the aliphatic hydrocarbon solvent flows out of the lower end opening of the second column 20, and is discarded (step 3). At this point, the organic halogen compound contained in the aliphatic hydrocarbon solvent from the adsorbent layer 100 is trapped by the trapping layer 200, and is separated from the aliphatic hydrocarbon solvent.

The trapping material contained in the trapping layer 200 has a great force of attracting a halogen group of the organic halogen compound, and therefore, can effectively trap the organic halogen compound regardless of the type and attribute thereof. That is, the transition metal held on the surface of the carrier of the trapping material has a d orbital with insufficient electrons. Meanwhile, in the organic halogen compound, electrons are biased to the halogen group. Thus, it is assumed that the transition metal easily forms, together with the halogen group of the organic halogen compound, an electron transfer complex and accordingly has a capacity of attracting and adsorbing the organic halogen compound. The capacity of the transition metal for attracting and adsorbing the organic halogen compound is further enhanced by the synergy with the carrier. That is, it is assumed as follows. Aluminum atoms (a Lewis acid site) on the surface of aluminum oxide contained in the carrier also lacks electrons, and therefore, aluminum oxide contained in the carrier attracts, to a carrier side, the electrons on the electron orbital of the transition metal. Accordingly, the transition metal further lacks the electrons on a surface layer side, and therefore, a force of attracting the halogen group increases. Consequently, a capacity of attracting and adsorbing the organic halogen compound is enhanced.

Part of the impurity contained in the aliphatic hydrocarbon solvent having passed through the adsorbent layer 100 passes, together with the solvent, through the trapping layer 200 and is discarded, and the other part of the impurity adsorbs to the trapping layer 200.

After the aliphatic hydrocarbon solvent has passed through the trapping layer 200, the upper end opening of the first column 10 is closed in an air-tight manner, and the solvent for extraction of the organic halogen compound is supplied to the second column 20 through the lower end opening thereof, and passes through the trapping layer 200 (step 4).

The extraction solvent used herein can be selected according to intended use of the organic halogen compound extract. For example, in a case where the organic halogen compound extract is used for analysis of the organic halogen compound, the extraction solvent can be selected according to a method for analyzing the organic halogen compound. Specifically, in a case where a gas chromatography method is employed as the analysis method, a solvent suitable for such a method, such as toluene or benzene, is preferably used as the extraction solvent. Alternatively, a solvent mixture obtained by addition of an aliphatic hydrocarbon solvent or an organochlorine solvent to toluene or benzene may be used. The aliphatic hydrocarbon solvent used in the solvent mixture is, for example, n-pentane, n-hexane, n-heptane, n-octane, iso-octane, or cyclohexane. The organochlorine solvent is, for example, dichloromethane, trichloromethane, or tetrachloromethane. In a case where a bioassay method is employed as the analysis method, a solvent suitable for such a method, such as a hydrophilic solvent including dimethylsulfoxide (DMSO) and methanol, is used.

The extraction solvent supplied to the trapping layer 200 extracts the organic halogen compound trapped by the trapping layer 200, and flows toward the branched path 22 of the second column 20 and is discharged from the branched path 22. The extraction solvent discharged from the branched path 22 as described above, i.e., the extraction solvent having passed through the trapping layer 200, is obtained, and the organic halogen compound extract is obtained accordingly (step 5).

In a case where the organic halogen compound-containing solution to which the above-described extraction method is applied is a solution containing polychlorinated biphenyls, various homologues of polychlorinated biphenyls with a chlorine number of 1 to 10 are trapped by the trapping layer 200, and are extracted from the trapping layer 200 by the extraction solvent. Thus, for a polychlorinated biphenyl extract obtained in the step 5, a failure to recover the homologues of polychlorinated biphenyls is suppressed. In a case where the organic halogen compound-containing solution contains dioxins (generally, a collective term of polychlorinated dibenzo-p-dioxins (PCDDs), polychlorinated dibenzofurans (PCDFs), and dioxin-like polychlorinated biphenyls (DL-PCBs); of 209 types of polychlorinated biphenyls (PCBs), the DL-PCBs are PCBs having toxicity similar to those of the PCDDs and the PCDFs and include non-ortho PCBs and mono-ortho PCBs) and polychlorinated biphenyls (non-DL-PCBs) not classified as dioxins, the PCDDs, the PCDFs, and various homologues of polychlorinated biphenyls with a chlorine number of 1 to 10 are trapped by the trapping layer 200, and are extracted from the trapping layer 200 by the extraction solvent. Thus, for the extract obtained in the step 5, a failure to recover the PCDDs, the PCDFs, and each homologue of polychlorinated biphenyls is suppressed.

Note that it has been known that in a case where the extract containing various dioxins and the non-DL-PCBs is analyzed using a high resolution gas chromatograph/mass spectrometer (a high resolution GC/MS), the mono-ortho PCBs influence a quantitative analysis result of the PCDDs and the PCDFs and the PCDDs and the PCDFs influence a quantitative analysis result of the mono-ortho PCBs. Thus, there is a probability that the analysis result lacks reliability. However, the reliability of the analysis result can be enhanced using a gas chromatograph/triple quadrupole mass spectrometer (GC-MS/MS).

### Second Embodiment

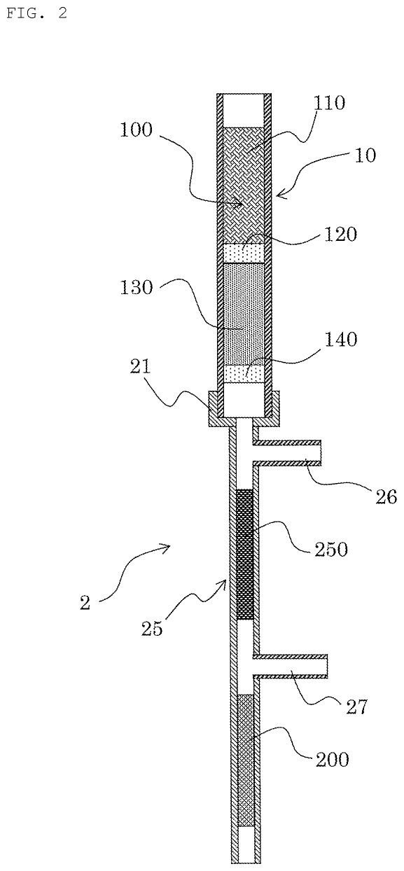

Another embodiment (a second embodiment) of an extraction column used for performing the method for extracting the organic halogen compound according to the present invention will be described with reference to FIG. 2. An extraction column 2 of the present embodiment is configured in such a manner that only the second column 20 of the extraction column 1 of the first embodiment is changed.

A second column 25 used in this embodiment is basically a cylindrical member opening at both ends, having a longer length than that of the second column 20 of the first embodiment, and made of a material similar to that of the first column 10. An attachment portion 21 similar to that of the first column 10 is formed on the upper end side of the second column 25 as viewed in the figure. The second column 25 has two branched paths opening at tip ends below the attachment portion 21, i.e., a first branched path 26 and a second branched path 27 spaced from each other.

The second column 25 is filled with a trapping layer 200 below the second branched path 27, and a portion between the first branched path 26 and the second branched path 27 is filled with a precedent trapping layer 250. The trapping layer 200 is a layer similar to the trapping layer 200 of the first column 20. The second column 25 is preferably set such that the inner diameter of the second column 25 is 3 to 10 mm, the length of a portion, which can be filled with the trapping layer 200, of the second column 25 is about 20 to 50 mm, and the length of a portion, which can be filled with the precedent trapping layer 250, of the second column 25 is about 20 to 50 mm.

The precedent trapping layer 250 is made of a carbon-based material or active magnesium silicate. The carbon-based material to be used may include, for example, activated carbon or graphite in the form of a grain and carbon material-containing silica gel such as activated carbon-containing silica gel or graphite-containing silica gel described in WO 2014/192055 A. The activity of active magnesium silicate is enhanced in such a manner that moisture is removed by heating of magnesium silicate, and such active magnesium silicate is described in JP-A-2020-115111. The carbon-based material to be used may also include a mixture of active magnesium silicate and graphite as described in JP-A-2020-115111.

Next, a method for extracting an organic halogen compound from an organic halogen compound-containing solution by means of the above-described extraction column 2 will be described. In this extraction method, the step 1 and the step 2 in the method for extracting the organic halogen compound by means of the extraction column 1 of the first embodiment are similarly executed.

In a step subsequent to the step 2, an aliphatic hydrocarbon solvent having flowed into the second column 25 from the first column 10 through an adsorbent layer 100 passes through the precedent trapping layer 250 and the trapping layer 200 in this order. Then, the aliphatic hydrocarbon solvent flows out of the lower end opening of the second column 25, and is discarded (step 3). At this point, the organic halogen compound contained in the aliphatic hydrocarbon solvent from the adsorbent layer 100 is trapped by each of the precedent trapping layer 250 and the trapping layer 200, and is separated from the aliphatic hydrocarbon solvent.

Part of an impurity contained in the aliphatic hydrocarbon solvent having passed through the adsorbent layer 100 passes, together with the aliphatic hydrocarbon solvent, through the precedent trapping layer 250 and the trapping layer 200 and is discarded, and the other part of the impurity is trapped by the precedent trapping layer 250 and the trapping layer 200.

Next, after the aliphatic hydrocarbon solvent has passed through the trapping layer 200, the upper end opening of the first column 10 and the opening of the first branched path 26 are closed in an air-tight manner, and a solvent for extraction of the organic halogen compound is supplied to the second column 25 through the lower end opening thereof and passes through the trapping layer 200 (step 4). The extraction solvent used herein is similar to that used in the first embodiment.

The extraction solvent supplied to the trapping layer 200 extracts the organic halogen compound trapped by the trapping layer 200, flows toward the second branched path 27 of the second column 25, and is discharged from the second branched path 27. The extraction solvent discharged from the second branched path 27 as described above, i.e., the extraction solvent having passed through the trapping layer 200, is obtained, and an extract containing the organic halogen compound trapped by the trapping layer 200 is obtained accordingly (step 5).

Next, the upper end opening of the first column 10 and the opening of the second branched path 27 are closed in an air-tight manner, and a solvent for extraction of the organic halogen compound is supplied to the second column 25 through the lower end opening thereof and passes through the trapping layer 200 and the precedent trapping layer 250 in this order (step 6). The extraction solvent used herein can be selected from solvents similar to those used in the first embodiment, and may be the same as or different from that used in the step 4.

The extraction solvent supplied to the precedent trapping layer 250 through the trapping layer 200 extracts the organic halogen compound trapped by the precedent trapping layer 250, flows toward the first branched path 26 of the second column 25, and is discharged from the first branched path 26. The extraction solvent discharged from the first branched path 26 as described above, i.e., the extraction solvent having passed through the precedent trapping layer 250, is obtained, and an extract containing the organic halogen compound trapped by the precedent trapping layer 250 is obtained accordingly (step 7).

In a case where the organic halogen compound-containing solution to which the above-described extraction method using the extraction column 2 is applied is a solution containing dioxins and polychlorinated biphenyls (non-DL-PCBs) which do not fall into the category of dioxins, non-ortho PCBs, PCDDs, and PCDFs of the dioxins are trapped by the precedent trapping layer 250, and mono-ortho PCBs of the dioxins and the non-DL-PCBs are trapped by the trapping layer 200. That is, the dioxins and the non-DL-PCBs contained in the organic halogen compound-containing solution are, in the second column 25, fractionated into a dioxin group including the non-ortho PCBs, the PCDDs, and the PCDFs trapped by the precedent trapping layer 250 and a PCB group including the mono-ortho PCBs and the non-DL-PCBs trapped by the trapping layer 200. Thus, an extract containing the above-described PCB group is obtained in the step 5, and an extract containing the above-described dioxin group is obtained in the step 7. The trapping layer 200 can trap various homologues of polychlorinated biphenyls with a chlorine number of 1 to 10, and therefore, for the PCB group extract obtained in the step 5, a failure to recover each homologue of polychlorinated biphenyls can be suppressed.

In this embodiment, the extract containing the above-described dioxin group and the extract containing the above-described PCB group can be separately obtained. Thus, each extract is analyzed using the high resolution GC/MS so that each component contained in the above-described dioxin group and each component contained in the above-described PCB group can be analyzed with a high accuracy.

### <Variations>

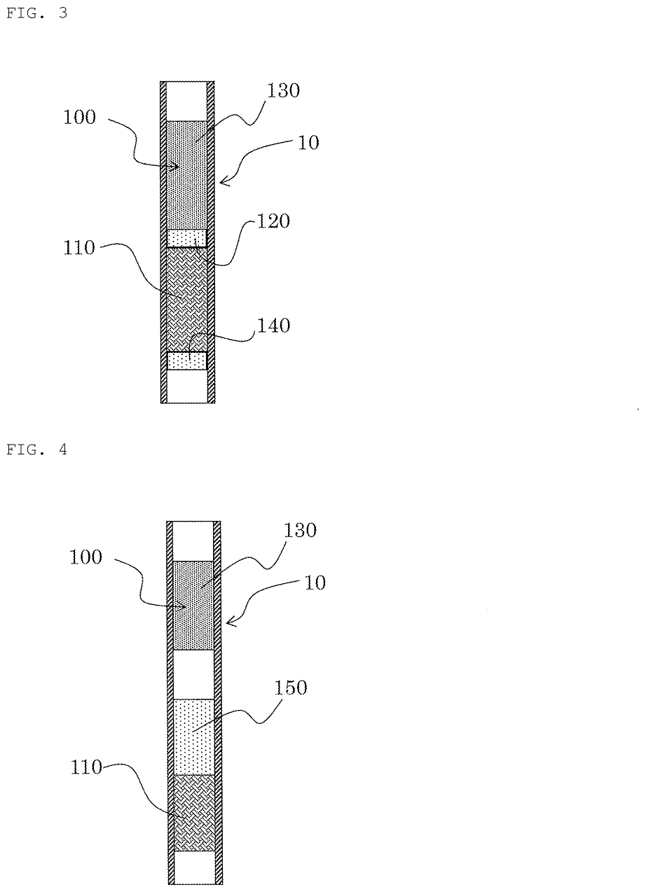

In each of the extraction columns 1, 2 of the first and second embodiments, various changes can be made to the adsorbent layer 100 of the first column 10. For example, as shown in FIG. 3, the order of the silver nitrate silica gel layer 110 and the sulfuric silica gel layer 130 may be switched. In this case, the impurity contained in the organic halogen compound-containing solution is decomposed mainly in the sulfuric silica gel layer 130, and the decomposition product thereof and part of the impurity are trapped by the silver nitrate silica gel layer 110. Both or one of the first active silica gel layer 120 and the second active silica gel layer 140 may be omitted. The adsorbent layer 100 may include only one of the silver nitrate silica gel layer 110 or the sulfuric silica gel layer 130.

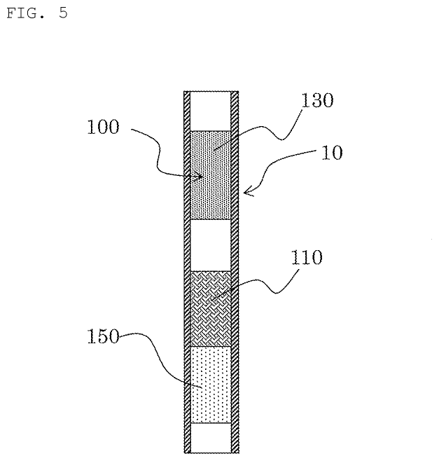

In a case where the order of the silver nitrate silica gel layer 110 and the sulfuric silica gel layer 130 is switched, a carrier layer 150 with fixed permanganate may be arranged between the sulfuric silica gel layer 130 and the silver nitrate silica gel layer 110, as shown in FIG. 4. In a case where the carrier layer 150 is arranged, sox gas generated in decomposition of the impurity in the sulfuric silica gel layer 130 can be consumed by the carrier layer 150, and therefore, the safety of an operation of extracting the organic halogen compound from the organic halogen compound-containing solution can be enhanced.

The carrier layer 150 is a layer formed in such a manner that permanganate is fixed to a carrier in the form of a grain, such as aluminum oxide, silica gel (normally, active silica gel of which the degree of activity has been enhanced by heating), crystalline aluminosilicate including zeolite, or any mixture thereof. Permanganate used herein is not specifically limited as long as permanganate is used as an oxidant, and for example, may include potassium permanganate, sodium permanganate, silver permanganate, magnesium permanganate, calcium permanganate, barium permanganate, and ammonium permanganate. A single type of permanganate may be used alone, or two or more types of permanganate may be used in combination.

The carrier layer 150 is prepared in such a manner that a permanganate aqueous solution is uniformly added to a surface of the carrier in the form of a grain with a particle size of about 10 to 500 μm and moisture is removed by heating under a reduced pressure such that a certain level of moisture content is maintained. The amount of permanganate fixed to the carrier is normally preferably set to at least 3% of the mass of the carrier and more preferably at least 4%. In a case where the fixed-permanganate amount is less than 3%, there is a probability that a capacity of consuming the sox gas generated in the course of decomposition of the impurity is degraded. A necessity of setting the upper limit of the amount of fixed permanganate is low because a capacity of consuming the SOx gas is enhanced by a great amount of fixed permanganate, but there is normally a limitation due to the degree of solubility of permanganate in the permanganate aqueous solution added to the carrier.

The moisture content of the carrier layer 150 is generally preferably set to 3 to 10% of the mass of the carrier and more preferably 4 to 6%. In a case where the moisture content is 3% or less, there is a probability that a capacity of consuming the sox gas generated in the course of decomposition of the impurity is significantly degraded. On the other hand, in a case where the moisture content exceeds 10%, there is a probability that the moisture content is increased due to action of moisture on the silver nitrate silica gel layer 110. As a result, there is a probability that an impurity decomposition effect is degraded.

The moisture content of the carrier layer 150 is preferably properly set according to the carrier. For example, in a case where the carrier is aluminum oxide, the moisture content of the carrier layer 150 is preferably set to 4 to 6% and more preferably 4.5 to 5%. In a case where the carrier is silica gel, the moisture content of the carrier layer 150 is preferably set to 3 to 20% and more preferably 4 to 10%, which is higher than that in the case where the carrier is aluminum oxide.

Because of an excellent impurity treatment capacity and suitability for treatment of a greater amount of organic halogen compound-containing solution, the carrier layer 150 in which aluminum oxide is used as the carrier and potassium permanganate is fixed to the carrier is preferably used. Preferably, the carrier layer 150 formed as follows is used: moisture and an adhering organic substance are removed by firing of aluminum oxide at 450 to 600° C. for 1 to 12 hours, the resultant aluminum oxide is injected into and uniformly mixed with a potassium permanganate aqueous solution prepared using ion-exchanged water or distilled water, and then, the resultant is dried using an evaporator such that the moisture content falls within the above-described range, for example.

Specifically, the carrier layer 150 formed as follows is preferably used: an aqueous solution in which potassium permanganate of 3 to 5% with respect to the mass of aluminum oxide to be injected is dissolved is used as the potassium permanganate aqueous solution and the amount of fixed potassium permanganate is adjusted to 3 to 5% of the mass of aluminum oxide.

In the carrier layer 150, the density of the carrier with fixed permanganate is not specifically limited, but is normally preferably set to 1.0 to 1.4 g/cm³ and more preferably 1.1 to 1.2 g/cm³. In a case where the density is less than 1.0 g/cm³, there is a probability that a capacity of consuming the sox gas generated in the course of decomposition of the impurity is degraded. Conversely, in a case where the density exceeds 1.4 g/cm³, the aliphatic hydrocarbon solvent supplied to the first column 10 is less likely to pass through the carrier layer 150, leading to a probability that an organic halogen compound-containing solution treatment efficiency is degraded.

In the case of using the first column 10 including the adsorbent layer 100 having the carrier layer 150, the aliphatic hydrocarbon solvent supplied to the adsorbent layer 100 in the step 2 penetrates and passes through the sulfuric silica gel layer 130. At this point, the aliphatic hydrocarbon solvent dissolves the organic halogen compound contained in the organic halogen compound-containing solution, the decomposition product of the impurity, the undecomposed remaining impurity, and the SOx gas generated in decomposition of the impurity, and then, an aliphatic hydrocarbon solvent solution containing the organic halogen compound flows toward and passes through the carrier layer 150.

The SOx gas contained in the aliphatic hydrocarbon solvent reacts with permanganate while the aliphatic hydrocarbon solvent is passing through the carrier layer 150, and is consumed accordingly. In a case where potassium permanganate is used as permanganate, the reaction is assumed to be as follows. Water (H₂O) involved in the reaction is moisture contained in the carrier layer 150.

2KMnO₄+5SO₂+2H₂O→2MnSO₄+2H₂SO₄+K₂SO₄ [Chemical Formula 1]

Meanwhile, the decomposition product and the impurity contained in the aliphatic hydrocarbon solvent from the sulfuric silica gel layer 130 are oxidized by permanganate while the aliphatic hydrocarbon solvent is passing through the carrier layer 150. In a case where the decomposition product and the impurity are, for example, unsaturated fatty acid or alkene (hydrocarbon with a double bond), these components are carbonylated through glycolization by oxidation action of permanganate, and part thereof is oxidized into carboxylic acid. In a case where the generated carboxylic acid is formic acid, such acid is further oxidized and decomposed into water and carbon dioxide.

The decomposition product and the impurity remaining in the aliphatic hydrocarbon solvent having passed through the carrier layer 150 are trapped by the silver nitrate silica gel layer 110 while the aliphatic hydrocarbon solvent is passing through the silver nitrate silica gel layer 110. As a result, the aliphatic hydrocarbon solvent having passed through the adsorbent layer 100 in the step 2 turns into an aliphatic hydrocarbon solvent solution containing the organic halogen compound contained in the organic halogen compound-containing solution, the SOx gas having been consumed and the decomposition product and the impurity having been significantly removed from the aliphatic hydrocarbon solvent solution.

The aliphatic hydrocarbon solvent can be supplied to the sulfuric silica gel layer 130 while being pressurized, as necessary. For example, the sulfuric silica gel layer 130 is sometimes clogged with the decomposition product due to the reaction between the sulfuric silica gel layer 130 and the impurity in the organic halogen compound-containing solution. Even in this case, the aliphatic hydrocarbon solvent stably and smoothly passes through the sulfuric silica gel layer 130 in such a manner that the aliphatic hydrocarbon solvent is supplied while being pressurized.

The adsorbent layer 100 having the carrier layer 150 may be configured such that the arrangement order of the carrier layer 150 and the silver nitrate silica gel layer 110 is switched. That is, as shown in FIG. 5, in the adsorbent layer 140, the silver nitrate silica gel layer 110 may be arranged on the upper layer side, and the carrier layer 150 may be arranged on the lower layer side.

In the case of using the adsorbent layer 100 according to this variation, part of the decomposition product and the impurity contained in the aliphatic hydrocarbon solvent having passed through the sulfuric silica gel layer 130 is trapped by the silver nitrate silica gel layer 110 while the aliphatic hydrocarbon solvent is passing through the silver nitrate silica gel layer 110, and the sox gas contained in the aliphatic hydrocarbon solvent generates NOx gas by reaction between part of the sox gas and silver nitrate. The decomposition product and the impurity remaining in the aliphatic hydrocarbon solvent having passed through the silver nitrate silica gel layer 110 are oxidized by permanganate while the aliphatic hydrocarbon solvent is passing through the carrier layer 150. In a case where the decomposition product and the impurity are, for example, unsaturated fatty acid or alkene (hydrocarbon with a double bond), these components are decomposed through the above-described oxidation process due to action of permanganate. Part of the decomposition product and other impurities remain in the carrier layer 150, and the remaining part is dissolved in the aliphatic hydrocarbon solvent and flows toward the second column 20 from the first column 10. Meanwhile, the sox gas and the NOx gas contained in the aliphatic hydrocarbon solvent from the silver nitrate silica gel layer 110 react with permanganate and are consumed while the aliphatic hydrocarbon solvent is passing through the carrier layer 150. In a case where potassium permanganate is used as permanganate, consumption reaction of the SOx gas by potassium permanganate is as already described above, and consumption reaction of the NOx gas by potassium permanganate is assumed to be according to (i) and (ii) below. Water (H₂O) involved in such reaction is moisture contained in the carrier layer 150.

[Chemical Formula 3]

3NO₂+H₂O→2HNO₃+NO (i)

2MnO₄⁻+6H⁺+5NO⁻→2Mn²⁺+5NO₂+3H₂O (ii)

In the reaction (ii), NO generated in the reaction (i) is consumed, and NO₂ is generated accordingly. The generated NO₂ is targeted for consumption in the reaction (i). Thus, it is assumed that by repeating the reaction (i) and the reaction (ii) in this order in the carrier layer 150 or progressing the reaction (i) and the reaction (ii) in parallel, consumption of the NOx gas progresses and the NOx gas gradually decreases and disappears.

As a result, the aliphatic hydrocarbon solvent having passed through the adsorbent layer 100 in the step 2 turns into an aliphatic hydrocarbon solvent solution storing and containing the organic halogen compound contained in the organic halogen compound-containing solution, the SOx gas and the NOx gas having been consumed and the decomposition product and the impurity having been significantly removed from the aliphatic hydrocarbon solvent solution.

In the above-described extraction column 1, 2, the second column 20, 25 does not necessarily have the branched path(s). In the case of using such a second column 20, 25, the steps 4 and 6 of the organic halogen compound extraction operation are executed with the second column 20, 25 detached from the first column 10.

Each figure as a reference in each of the above-described embodiments shows the outline of the extraction column 1, 2 or the first column 10, and does not precisely reflect the structure, shape, size, ratio and the like of each component.

### EXAMPLES

Hereinafter, the present invention will be specifically described with reference to examples and the like, but is not limited to these examples and the like. Fillers, organic halogen compound-containing solutions, internal standard substances, and extraction columns used in the following examples and comparative examples are as follows.

### [Filler]

### Silver Nitrate Silica Gel:

An aqueous solution in which silver nitrate (a product name “Silver Nitrate” 198-00835 manufactured by FUJIFILM Wako Pure Chemical Corporation, a special reagent grade) is dissolved in distilled water was added to and uniformly mixed with active silica gel (manufactured by KANTO CHEMICAL CO., INC.). Silver nitrate silica gel was used, which was prepared in such a manner that the resultant mixture is heated to 70° C. under a reduced pressure by means of a rotary evaporator and is dried. A silver nitrate aqueous solution of which the amount of silver nitrate with respect to the mass of the active silica gel is set to 10% was used, and the amount of silver nitrate in the silver nitrate silica gel was set to 10% of the mass of the active silica gel.

### Sulfuric Silica Gel:

Sulfuric silica gel was used, which was prepared in such a manner that concentrated sulfuric acid (a product name “Sulfuric Acid” 190-04675 manufactured by FUJIFILM Wako Pure Chemical Corporation, for precision analysis) is uniformly added to active silica gel (manufactured by KANTO CHEMICAL CO., INC.) and the resultant is dried. The amount of concentrated sulfuric acid added to the active silica gel was set such that the amount of sulfuric acid with respect to the active silica gel is 44% in terms of mass.

### Silver-Supporting Aluminum Oxide A:

A silver nitrate aqueous solution was prepared in such a manner that silver nitrate (a product name “Silver Nitrate” 198-00835 manufactured by FUJIFILM Wako Pure Chemical Corporation, a special reagent grade) of 17 g is weighed in a beaker with a capacity of 300 mL and ion-exchanged water of 100 mL is added thereto. A sodium hydroxide aqueous solution was prepared in such a manner that sodium hydroxide (“Sodium Hydroxide, Granular” 198-13765 manufactured by FUJIFILM Wako Pure Chemical Corporation, a special reagent grade 97.0%) of 4.1 g is dissolved in ion-exchanged water of 20 mL. Aluminum oxide (a product name “Aluminium Oxide 90 active basic-(activity stage I) for column chromatography” manufactured by Merck KGaA/Particle Size: 0.063 to 0.200 mm) of 50 g was added to and sufficiently mixed with the silver nitrate aqueous solution, and thereafter, the resultant was left to stand for about 30 minutes. Aluminum oxide used herein was subjected to drying by heating to 300° C. in an oven in advance. After the mixture had been left to stand, the resultant was sufficiently mixed again. Then, the total amount of sodium hydroxide aqueous solution was added to and promptly sufficiently mixed with the resultant. For the resultant solution, suction filtration was performed using a Buchner funnel provided with a glass fiber paper filter (GA-55 manufactured by ADVANTEC CO., LTD.), and moisture in a filtration residue was roughly filtrated in such a manner that a suction state is maintained for about 5 minutes. The filtration residue was returned to the beaker, and was rinsed in such a manner that ion-exchanged water of 100 mL is added to and sufficiently mixed with the filtration residue. For the resultant filtration residue, suction filtration was similarly performed again, and moisture in the filtration residue was roughly filtrated in such the manner that the suction state is maintained for about 5 minutes. The resultant filtration residue was transferred to an evaporation dish (a firing dish), was spread with a uniform thickness, and was dried in the oven at 120° C. for 12 hours. The dried filtration residue was placed in a firing silica tube, and was fired using a tubular furnace at 700° C. for 2 hours under a nitrogen stream of 1.0 to 1.2 L/min. The resultant silver-supporting aluminum oxide was used as silver-supporting aluminum oxide A.

### Silver-Supporting Aluminum Oxide B:

A silver nitrate aqueous solution was prepared in such a manner that silver nitrate (a product name “Silver Nitrate” 198-00835 manufactured by FUJIFILM Wako Pure Chemical Corporation, a special reagent grade) of 22.8 g is weighed in a beaker with a capacity of 500 mL and ion-exchanged water of 200 mL is added thereto. A sodium hydroxide aqueous solution was prepared in such a manner that sodium hydroxide (“Sodium Hydroxide, Granular” 198-13765 manufactured by FUJIFILM Wako Pure Chemical Corporation, a special reagent grade 97.0%) of 5.36 g is dissolved in ion-exchanged water of 80 mL. Aluminum oxide (a product name “Aluminium Oxide 90 active basic-(activity stage I) for column chromatography” manufactured by Merck KGAA/Particle Size: 0.063 to 0.200 mm) of 200 g was added to and sufficiently mixed with the silver nitrate aqueous solution, and thereafter, the resultant was left to stand for about 30 minutes. Aluminum oxide used herein was subjected to drying by heating to 300° C. in an oven in advance. After the mixture had been left to stand, the resultant was sufficiently mixed again. Then, the total amount of sodium hydroxide aqueous solution was added to and promptly sufficiently mixed with the resultant. For the resultant solution, suction filtration was performed using a Buchner funnel provided with a glass fiber paper filter (GA-55 manufactured by ADVANTEC CO., LTD.), and moisture in a filtration residue was roughly filtrated in such a manner that a suction state is maintained for about 5 minutes. The filtration residue was returned to the beaker, and was rinsed in such a manner that ion-exchanged water of 200 mL is added to and sufficiently mixed with the filtration residue. For the resultant filtration residue, suction filtration was similarly performed again, and moisture in the filtration residue was roughly filtrated in such the manner that the suction state is maintained for about 5 minutes. The resultant filtration residue was transferred to an evaporation dish (a firing dish), was spread with a uniform thickness, and was dried in the oven at 120° C. for 24 hours. The dried filtration residue was placed in a firing silica tube, and was fired using a tubular furnace at 700° C. for 2 hours under a nitrogen stream of 1.5 to 3.0 L/min. The resultant silver-supporting aluminum oxide was used as silver-supporting aluminum oxide B.

### Silver Oxide-Modified Aluminum Oxide:

A silver nitrate aqueous solution was prepared in such a manner that silver nitrate (a product name “Silver Nitrate” 198-00835 manufactured by FUJIFILM Wako Pure Chemical Corporation, a special reagent grade) of 56 g is weighed in a beaker with a capacity of 600 mL and ion-exchanged water of 100 mL is added thereto. Aluminum oxide (a product name “Aluminium Oxide 90 active basic-(activity stage I) for column chromatography” manufactured by Merck KGAA/Particle Size: 0.063 to 0.200 mm) of 164 g was placed in an eggplant flask, and the total amount of silver nitrate aqueous solution was added thereto and was sufficiently mixed therewith. Thereafter, the resultant was left to stand for about 60 hours. After the resultant had been left to stand, the resultant was sufficiently mixed again. Then, the eggplant flask was attached to a rotary evaporator, and the resultant was dried such that the moisture content of the contents in the eggplant flask reaches 1.5% or less under conditions of a hot-water bath temperature of 70° C., a rotation number of 30 to 100 rpm, and 60 hPa. Dried contents of 50 g was transferred to a firing silica tube, and was fired using a tubular furnace at 650° C. for 2.53 hours under a nitrogen stream of 3.0 L/min. The resultant silver oxide-modified aluminum oxide was used.

### Graphite-Mixed Active Magnesium Silicate:

A mixture was prepared in such a manner that graphite (a product name “ENVI-Carb” manufactured by Sigma-Aldrich) is added to and uniformly mixed with magnesium silicate (a product name “Florisil, 75 to 150 μm” manufactured by FUJIFILM Wako Pure Chemical Corporation). The percentage of mixed graphite in the mixture was set to 12.5 mass %. The resultant mixture was heated at 450° C. for 2 hours in a tubular furnace under a nitrogen stream set to a flow rate of 0.5 to 1.0 L/min. The resultant graphite-mixed active magnesium silicate was used.

### Aluminum Oxide:

Three types of a product name “Aluminium Oxide 90 active basic-(activity stage I) for column chromatography” manufactured by Merck KGAA and having different manufacturing lots (a particle size of 0.063 to 0.200 mm) were used.

### [Organic Halogen Compound-Containing Solution]

PCB Surrogate Standard Solution: A product name “PCB-LCS-H” manufactured by Wellington Laboratories Inc. and containing homologues of polychlorinated biphenyls labelled by ¹³C₁₂ and having IUPAC numbers of #1, #3, #4, #15, #19, #28, #54, #52, #70, #81, #77, #104, #95, #101, #123, #118, #114, #105, #126, #155, #153, #138, #167, #156, #157, #169, #188, #180, #170, #189, #202, #205, #208, and #209 was diluted 100-fold with decane, and the resultant was used.

### Dioxin Surrogate Standard Solution:

A product name “DF-LCS-A” manufactured by Wellington Laboratories Inc. and containing 17 types of homologues of PCDDs and PCDFs labelled by ¹³C₁₂ was diluted 100-fold with decane, and the resultant was used.

### Fish Oil:

Fish oil similar to one used in a simultaneous dioxin analysis accuracy control test (proficiency testing) performed in 2016 in EU-reference laboratories (EU-RL) was used.

### Sunflower Oil:

Commercially-available sunflower oil was used.

### Bottom Sediment Extract:

A bottom sediment sample collected from a river in Japan was dried with air, and a toluene solution was obtained using toluene by application of a Soxhlet extraction method to a bottom sediment sample of 10 g. The volume of the toluene solution was fixed at 20 mL.

### Soil Extract:

A soil sample collected in Japan was dried with air, and a toluene solution was obtained using toluene by application of a Soxhlet extraction method to a soil sample of 10 g. The volume of the toluene solution was fixed at 20 mL.

### Beef Tallow Extract:

A product name “Beef Tallow” 021-00515 manufactured by FUJIFILM Wako Pure Chemical Corporation was used.

### [Internal Standard Substance]

### PCB Internal Standard Substance:

A product manufactured by Wellington Laboratories Inc. and containing homologues of polychlorinated biphenyls labelled by ¹³C₁₂ and having IUPAC numbers of #9, #37, #79, #111, #162, #178, #194, and #206 was used.

### Dioxin Internal Standard Substance:

A product manufactured by Wellington Laboratories Inc. and containing four types of homologues of PCDDs labelled by ¹³C₁₂ was used.

### [Extraction Column]

### Extraction Column A:

The extraction column 1 of the first embodiment shown in FIG. 1 was used, the specifications of each component being set as follows.

### First Column 10

In the first column 10 set to an outer diameter of 18.5 mm, an inner diameter of 12.5 mm, and a length of 200 mm, silver nitrate silica gel of 4.4 g (a filling height of 60 mm) was stacked on sulfuric silica gel of 8.5 g (a filling height of 80 mm), and in this manner, the adsorbent layer 100 was formed (no first active silica gel layer and no second active silica gel layer were stacked).

### Second Column 20

In the second column 20 set to an outer diameter of 8 mm, an inner diameter of 6 mm, and a length of 50 mm, the second column 20 was filled with silver-supporting aluminum oxide A of 0.75 g such that the filling height reaches 28 mm, and in this manner, the trapping layer 200 was formed.

### Extraction Column B:

An extraction column set similarly to the extraction column A except that the trapping layer 200 was formed using the silver-supporting aluminum oxide B instead of the silver-supporting aluminum oxide A was used.

### Extraction Column C:

An extraction column set similarly to the extraction column A except that the trapping layer 200 was formed using the silver oxide-modified aluminum oxide instead of the silver-supporting aluminum oxide A was used.

### Extraction Column D:

The extraction column 2 of the second embodiment shown in FIG. 2 was used, the specifications of each component being set as follows.

### First Column 10

The first column 10 was set similarly to the first column 10 of the extraction column A.

### Second Column 25

In the second column 25 set to an outer diameter of 8 mm, an inner diameter of 6 mm, and a length of 130 mm, the precedent trapping layer 250 was formed in such a manner that the second column 25 is filled with graphite-mixed active magnesium silicate of 0.43 g such that the filling height reaches 28 mm, and the trapping layer 200 was formed in such a manner that the second column 25 is filled with silver-supporting aluminum oxide A of 0.75 g such that the filling height reaches 28 mm.

### Extraction Column E:

An extraction column set similarly to the extraction column D except that the trapping layer 200 was formed using the silver oxide-modified aluminum oxide instead of the silver-supporting aluminum oxide A was used.

### Extraction Column F:

Three types of extraction columns F1, F2, F3 set similarly to the extraction column A except that the trapping layer 200 was formed using aluminum oxide instead of the silver-supporting aluminum oxide A were produced and used. A difference among these extraction columns is a difference in a manufacturing lot for aluminum oxide to be used.

### Example 1 and Comparative Examples 1 to 4

A test solution (hereinafter referred to as a “standard solution”) was prepared in such a manner that n-hexane of 2 mL is placed in a test tube and the PCB surrogate standard solution and the dioxin surrogate standard solution are added thereto and sufficiently mixed therewith.

After n-hexane of 5 mL had been added to the first column of the extraction column shown in Table 1 and had infiltrated the adsorbent layer, the total amount of standard solution was added to the adsorbent layer. The test tube in which the standard solution was prepared was rinsed twice with n-hexane of 2 mL, and a rinse solution was also added to the adsorbent layer. Next, n-hexane of 2 mL was further added to the adsorbent layer, and thereafter, the entirety of the silver nitrate silica gel layer and the upper half of the sulfuric silica gel layer in the adsorbent layer were heated to 60° C. Then, n-hexane of 85 mL was gradually supplied to the adsorbent layer, and passed through the adsorbent layer and the trapping layer in this order. After n-hexane had passed through the trapping layer, the trapping layer was dried with passing compressed air. Then, the trapping layer was heated to 90° C., and thereafter, the upper opening of the first column was closed in an air-tight manner and toluene of 1.5 mL was supplied to the trapping layer through the lower opening of the second column. Then, toluene having passed through the trapping layer was recovered as an extract through the branched path. A time required until the extract is obtained after addition of the standard solution was about 1.5 hours.

The recovered extract was concentrated to 20 μL, and the PCB internal standard substance shown in Table 1 was added thereto. Then, the final volume of the extract was fixed at 50 μL. Polychlorinated biphenyl (PCB) contained in the extract was quantitatively analyzed by a GC-HRMS method, and the rate of recovery of each homologue of the PCB was calculated. Results are shown in Table 1. The rate of recovery of the homologue indicates the percentage (%) of the amount of homologue contained in collected toluene with respect to the initial amount of homologue contained in the test solution (the standard solution). The same also applies to the following other examples and comparative examples. Moreover, “—” in each table subsequent to Table 1 indicates an unmeasured item.

TABLE 1

| | Example | Comparative | Comparative | Comparative | Comparative |
|---|---|---|---|---|---|
| | 1 | Example 1 | Example 2 | Example 3 | Example 4 |
| PCB | Test Solution | | | | |
| Homologue | Standard | Standard | Standard | Standard | Standard |
| (Chlorine | Solution | Solution | Solution | Solution | Solution |
| Number - | Extraction Column | | | | |
| IUPAC | A | C | F1 | F2 | F3 |
| Number) | Rate of Recovery of PCB | | | | |
| 1Cl-#1 | 93% | 100% | — | — | — |
| 1Cl-#3 | 46% | 63% | 11% | 9% | 7% |
| 2Cl-#4 | 96% | 110% | — | — | — |
| 2Cl-#15 | 98% | 97% | — | — | — |
| 3Cl-#19 | 105% | 112% | — | — | — |
| 3Cl-#28 | 108% | 93% | 118% | 116% | 105% |
| 4Cl-#54 | 97% | 95% | — | — | — |
| 4Cl-#52 | 109% | 98% | 108% | 103% | 94% |
| 4Cl-#70 | 102% | 101% | — | — | — |
| 4Cl-#81 | 103% | 105% | 3% | 1% | 2% |
| 4Cl-#77 | 102% | 107% | 1% | 1% | 0% |
| 5Cl-#104 | 100% | 99% | — | — | — |
| 5Cl-#95 | 96% | 89% | — | — | — |
| 5Cl-#101 | 110% | 106% | 91% | 99% | 87% |
| 5Cl-#123 | 106% | 95% | 94% | 91% | 85% |
| 5Cl-#118 | 101% | 102% | 93% | 93% | 88% |
| 5Cl-#114 | 104% | 101% | 98% | 98% | 92% |
| 5Cl-#105 | 95% | 97% | 91% | 93% | 87% |
| 5Cl-#126 | 88% | 98% | 3% | 3% | 3% |
| 6Cl-#155 | 103% | 104% | — | — | — |
| 6Cl-#153 | 95% | 95% | 72% | 98% | 93% |
| 6Cl-#138 | 96% | 86% | 100% | 97% | 89% |
| 6Cl-#167 | 101% | 100% | 95% | 93% | 87% |
| 6Cl-#156 | 104% | 101% | 89% | 88% | 83% |
| 6Cl-#157 | 95% | 95% | 87% | 87% | 83% |
| 6Cl-#169 | 95% | 98% | 7% | 6% | 7% |
| 7Cl-#188 | 103% | 93% | — | — | — |
| 7Cl-#180 | 100% | 109% | 74% | 91% | 91% |
| 7Cl-#170 | 95% | 111% | 92% | 89% | 87% |
| 7Cl-#189 | 100% | 102% | 89% | 86% | 86% |
| 8Cl-#202 | 103% | 91% | — | — | — |
| 8Cl-#194 | — | — | 80% | 97% | 87% |
| 8Cl-#205 | 101% | 98% | — | — | — |
| 9Cl-#206 | — | — | 18% | 43% | 74% |
| 9Cl-#208 | 108% | 103% | — | — | — |
| 10Cl-#209 | 66% | 28% | 0% | 0% | 0% |

According to Table 1, the rate of recovery is low for some of the PCBs with a chlorine number of 1 and the PCB with a chlorine number of 10 in Comparative Examples 1 to 4, whereas is high for all homologues of the PCBs including the PCBs with chlorine numbers of 1 and 10 in Example 1. This shows as follows: in Comparative Examples 1 to 4, some of the homologues of the PCB are less likely to be trapped by the trapping layer in the course of extraction, and for this reason, a failure to recover some homologues is caused in the course of extraction; and on the other hand, in Example 1, such a failure is less likely to be caused. Note that in any of Comparative Examples 2 to 4, the extraction column F was used, but there is a great difference in the rate of recovery among some PCB homologues (typically, #206) due to a difference in the manufacturing lot of aluminum oxide used for the trapping layer. This shows that Comparative Examples 2 to 4 are inferior in terms of the stability and reliability of the PCB extraction results.

### Examples 2 and 3 and Comparative Example 5

A test solution (hereinafter referred to as a “fish oil sample” or a “sunflower oil sample”) was prepared in such a manner that the fish oil or the sunflower oil is weighed in a test tube, n-hexane of an amount twice as much as the volume of the fish oil or the sunflower oil is added thereto, and then, the PCB surrogate standard solution is further added to and sufficiently mixed with the resultant. Then, from the fish oil sample or the sunflower oil sample, an extract was recovered by steps similar to those of Example 1, and was analyzed in a manner similar to that of Example 1. In this manner, the rate of recovery of each homologue of the PCB was calculated. Results are shown in Table 2. Note that a time required until the extract is obtained after addition of the test solution was about 1.5 hours.

TABLE 2

| | Example | Example | Comparative |
|---|---|---|---|
| | 2 | 3 | Example 5 |
| PCB | Test Solution | | |
| Homologue | Fish Oil | Sunflower | Sunflower |
| (Chlorine | Sample | Oil Sample | Oil Sample |
| Number - | Extraction Column | | |
| IUPAC | A | B | C |
| Number) | Rate of Recovery of PCB | | |
| 1Cl-#1 | 95% | 83% | 77% |
| 1Cl-#3 | 58% | 25% | 0% |
| 2Cl-#4 | 100% | 99% | 93% |
| 2Cl-#15 | 102% | 82% | 0% |
| 3Cl-#19 | 94% | 103% | 92% |
| 3Cl-#28 | 112% | 88% | 80% |
| 4Cl-#54 | 96% | 90% | 80% |
| 4Cl-#52 | 91% | 96% | 82% |
| 4Cl-#70 | 102% | 101% | 89% |
| 4Cl-#81 | 104% | 86% | 0% |
| 4Cl-#77 | 102% | 84% | 0% |
| 5Cl-#104 | 99% | 98% | 86% |
| 5Cl-#95 | 87% | 97% | 87% |
| 5Cl-#101 | 100% | 96% | 94% |
| 5Cl-#123 | 102% | 89% | 94% |
| 5Cl-#118 | 96% | 87% | 88% |
| 5Cl-#114 | 109% | 93% | 95% |
| 5Cl-#105 | 105% | 85% | 89% |
| 5Cl-#126 | 109% | 92% | 0% |
| 6Cl-#155 | 92% | 95% | 89% |
| 6Cl-#153 | 95% | 102% | 98% |
| 6Cl-#138 | 99% | 97% | 95% |
| 6Cl-#167 | 100% | 98% | 90% |
| 6Cl-#156 | 102% | 103% | 87% |
| 6Cl-#157 | 99% | 100% | 86% |
| 6Cl-#169 | 100% | 101% | 0% |
| 7Cl-#188 | 86% | 99% | 86% |
| 7Cl-#180 | 95% | 103% | 98% |
| 7Cl-#170 | 91% | 99% | 99% |
| 7Cl-#189 | 105% | 115% | 101% |
| 8Cl-#202 | 93% | 98% | 94% |
| 8Cl-#194 | — | — | — |
| 8Cl-#205 | 92% | 112% | 97% |
| 9Cl-#206 | — | — | — |
| 9Cl-#208 | 101% | 101% | 101% |
| 10Cl-#209 | 44% | 76% | 65% |

According to Table 2, the rate of recovery is high for all homologues of the PCBs including the PCBs with chlorine numbers of 1 and 10 in Examples 2 and 3. This shows that a failure to recover the homologues of the PCB in the course of extraction is less caused.

### Examples 4 and 5 and Comparative Examples 6 and 7

A test solution (a bottom sediment extract sample or a soil extract sample) was prepared in such a manner that the PCB surrogate standard solution is added to and sufficiently mixed with the bottom sediment extract sample or the soil extract sample to form a solution, toluene is removed using a rotary evaporator from the solution, and n-hexane of 1 mL is added to and dissolved in the residue. From the test solution, an extract was recovered by steps similar to those of Example 1, and was analyzed in a manner similar to that of Example 1. In this manner, the rate of recovery of each homologue of the PCB was calculated. Results are shown in Table 3. Note that a time required until the extract is obtained after addition of the test solution was about 1.5 hours.

TABLE 3

| | Example | Comparative | Example | Comparative |
|---|---|---|---|---|
| | 4 | Example 6 | 5 | Example 7 |
| | Test Solution | | | |
| | Bottom | Bottom | | |
| PCB | Sediment | Sediment | Soil | Soil |
| Homologue | Extract | Extract | Extract | Extract |
| (Chlorine | Sample | Sample | Sample | Sample |
| Number - | Extraction Column | | | |
| IUPAC | B | C | B | C |
| Number) | Rate of Recovery of PCB | | | |
| 1Cl-#1 | 81% | — | 77% | — |
| 1Cl-#3 | 42% | 0% | 36% | 0% |
| 2Cl-#4 | 96% | — | 93% | — |
| 2Cl-#15 | 82% | — | 79% | — |
| 3Cl-#19 | 91% | — | 100% | — |
| 3Cl-#28 | 89% | 92% | 88% | 88% |
| 4Cl-#54 | 86% | — | 85% | — |
| 4Cl-#52 | 88% | 92% | 82% | 85% |
| 4Cl-#70 | 90% | — | 87% | — |
| 4Cl-#81 | 93% | 0% | 88% | 0% |
| 4Cl-#77 | 89% | 0% | 88% | 0% |
| 5Cl-#104 | 88% | — | 87% | — |
| 5Cl-#95 | 86% | — | 91% | — |
| 5Cl-#101 | 91% | 80% | 111% | 88% |
| 5Cl-#123 | 97% | 100% | 101% | 88% |
| 5Cl-#118 | 90% | 103% | 72% | 93% |
| 5Cl-#114 | 90% | 96% | 93% | 86% |
| 5Cl-#105 | 98% | 92% | 49% | 85% |
| 5Cl-#126 | 90% | 0% | 99% | 0% |
| 6Cl-#155 | 89% | — | 87% | — |
| 6Cl-#153 | 91% | 85% | 95% | 92% |
| 6Cl-#138 | 92% | 82% | 104% | 89% |
| 6Cl-#167 | 96% | 99% | 93% | 89% |
| 6Cl-#156 | 94% | 101% | 111% | 92% |
| 6Cl-#157 | 94% | 105% | 91% | 92% |
| 6Cl-#169 | 98% | 0% | 97% | 0% |
| 7Cl-#188 | 93% | — | 102% | — |
| 7Cl-#180 | 93% | 100% | 90% | 96% |
| 7Cl-#170 | 94% | 96% | 85% | 86% |
| 7Cl-#189 | 98% | 115% | 104% | 102% |
| 8Cl-#202 | 94% | — | 89% | — |
| 8Cl-#194 | — | — | — | — |
| 8Cl-#205 | 93% | — | 96% | — |
| 9Cl-#206 | — | — | — | — |
| 9Cl-#208 | 96% | — | 90% | — |
| 10Cl-#209 | 76% | 24% | 65% | 1% |

According to Table 3, the rate of recovery is low for a considerable number of the PCB homologues in Comparative Examples 6 and 7, whereas is high for each homologue with a chlorine number of 1 to 10 in Examples 4 and 5. This shows as follows: in Comparative Examples 6 and 7, some of the homologues of the PCB are less likely to be trapped by the trapping layer, and for this reason, a failure to recover some homologues is caused in the course of extraction; and on the other hand, in Examples 4 and 5, such a failure is less likely to be caused.

### Example 6 and Comparative Example 8

A test solution (hereinafter referred to as a “beef tallow sample”) was prepared in such a manner that a beef tallow extract is weighed in a test tube, n-hexane of an amount twice as much as the volume of the beef tallow extract is added thereto, and the PCB surrogate standard solution and the dioxin surrogate standard solution are added to and sufficiently mixed with the resultant.

After n-hexane of 5 mL had been added to the first column of the extraction column shown in Table 4 and had infiltrated the adsorbent layer, the total amount of beef tallow sample was added to the adsorbent layer. The test tube in which the beef tallow sample was prepared was rinsed twice with n-hexane of 2 mL, and a rinse solution was also added to the adsorbent layer. Next, n-hexane of 2 mL was further added to the adsorbent layer, and thereafter, the entirety of the silver nitrate silica gel layer and the upper half of the sulfuric silica gel layer in the adsorbent layer were heated to 60° C. Then, n-hexane of 85 mL was gradually supplied to the adsorbent layer, and passed through the adsorbent layer, the precedent trapping layer, and the trapping layer in this order. After n-hexane had passed through the trapping layer, the trapping layer and the precedent trapping layer were dried with passing compressed air.

Then, the trapping layer was heated to 90° C., and thereafter, the upper opening of the first column and the first branched path of the second column were closed in an air-tight manner and toluene of 1.0 mL was supplied to the trapping layer through the lower opening of the second column. Then, toluene having passed through the trapping layer was recovered through the second branched path, and in this manner, a first extract was obtained.

Subsequently, the precedent trapping layer was heated to 90° C., and thereafter, the upper opening of the first column and the second branched path of the second column were closed in an air-tight manner and toluene of 1.5 mL was supplied to the trapping layer through the lower opening of the second column. Then, toluene having passed through the trapping layer and the precedent trapping layer in this order was recovered through the first branched path, and in this manner, a second extract was obtained. A time required until the second extract is obtained after addition of the beef tallow sample was about 1.5 hours.

The first extract was concentrated to 20 μL, and the PCB internal standard substance was added thereto. Then, the final volume of the first extract was fixed at 50 μL. Polychlorinated biphenyl (PCB) contained in the first extract was quantitatively analyzed by the GC-HRMS method, and the rate of recovery of each homologue of the PCB was calculated. In addition, the second extract was concentrated to 10 μL, and the dioxin internal standard substance was added thereto. Then, the final volume of the second extract was fixed at 20 μL. Dioxin-like polychlorinated biphenyl (DL-PCBs) contained in the second extract was quantitatively analyzed by the GC-HRMS method, and the rate of recovery of each homologue of the DL-PCBs was calculated. Results are shown in Table 4.

TABLE 4

| | | Comparative |
|---|---|---|
| | Example 6 | Example 8 |
| | Test Solution | |
| PCB | Beef Tallow Sample | Beef Tallow Sample |
| Homologue | Extraction Column | |
| (Chlorine | D | E |

| | |
|---|---|
| Number | Rate of Recovery of PCB |

| IUPAC | Second | First | Second | First |
|---|---|---|---|---|
| Number) | Extract | Extract | Extract | Extract |
| 1Cl-#1 | 0% | 87% | 0% | 90% |
| 1Cl-#3 | 25% | 0% | 26% | 12% |
| 2Cl-#4 | 0% | 98% | 0% | 102% |
| 2Cl-#15 | 83% | 0% | 93% | 0% |
| 3Cl-#19 | 0% | 97% | 0% | 101% |
| 3Cl-#28 | 5% | 85% | 2% | 98% |
| 4Cl-#54 | 0% | 87% | 1% | 98% |
| 4Cl-#52 | 0% | 92% | 1% | 102% |
| 4Cl-#70 | 4% | 91% | 1% | 99% |
| 4Cl-#81 | 84% | 0% | 95% | 1% |
| 4Cl-#77 | 83% | 0% | 98% | 1% |
| 5Cl-#104 | 0% | 91% | 2% | 107% |
| 5Cl-#95 | 0% | 93% | 1% | 103% |
| 5Cl-#101 | 0% | 101% | 1% | 95% |
| 5Cl-#123 | 0% | 92% | 2% | 98% |
| 5Cl-#118 | 0% | 89% | 3% | 105% |
| 5Cl-#114 | 0% | 96% | 1% | 102% |
| 5Cl-#105 | 0% | 88% | 16% | 83% |
| 5Cl-#126 | 92% | 0% | 91% | 3% |
| 6Cl-#155 | 0% | 95% | 1% | 97% |
| 6Cl-#153 | 0% | 104% | 1% | 104% |
| 6Cl-#138 | 0% | 103% | 1% | 86% |
| 6Cl-#167 | 0% | 89% | 2% | 101% |
| 6Cl-#156 | 0% | 100% | 1% | 97% |
| 6Cl-#157 | 0% | 96% | 1% | 94% |
| 6Cl-#169 | 99% | 0% | 99% | 1% |
| 7Cl-#188 | 0% | 108% | 1% | 100% |
| 7Cl-#180 | 0% | 102% | 1% | 104% |
| 7Cl-#170 | 0% | 105% | 1% | 101% |
| 7Cl-#189 | 0% | 118% | 1% | 102% |
| 8Cl-#202 | 0% | 94% | — | — |
| 8Cl-#205 | 0% | 111% | 1% | 97% |
| 9Cl-#208 | 0% | 81% | 1% | 103% |
| 10Cl-#209 | 1% | 69% | 0% | 34% |

Table 4 shows that in Example 6, the homologues of the PCB with a chlorine number of 1 to 10 in the beef tallow sample are fractionated into the second extract mainly with non-ortho PCBs and the first extract mainly with mono-ortho PCBs and non-DL-PCBs and the rate of recovery is high for any of these homologues. On the other hand, in Comparative Example 8, the homologues of the PCB are fractionated into the first extract and the second extract as in Example 6, but the rate of recovery of the PCB with a chlorine number of 10 is particularly low. This shows as follows: in Comparative Example 8, some of the PCB homologues are less likely to be trapped by the precedent trapping layer or the trapping layer, and for this reason, a failure to recover some homologues is caused in the course of extraction; and on the other hand, in Example 6, such a failure is less likely to be caused.

### LIST OF REFERENCE NUMERALS

1, 2 Extraction Column
10 First Column
20, 25 Second Column
100 Adsorbent Layer
200 Trapping Layer
250 Precedent Trapping Layer

1. A method for extracting an organic halogen compound from a solution containing the organic halogen compound and an impurity, comprising:
a step 1 of adding the solution to an adsorbent layer capable of treating the impurity;
a step 2 of supplying an aliphatic hydrocarbon solvent to the adsorbent layer to which the solution has been added and causing the aliphatic hydrocarbon solvent to pass through the adsorbent layer;
a step 3 of supplying the aliphatic hydrocarbon solvent having passed through the adsorbent layer to a trapping layer capable of trapping the organic halogen compound and causing the aliphatic hydrocarbon solvent to pass through the trapping layer;
a step 4 of supplying a solvent for extraction of the organic halogen compound to the trapping layer through which the aliphatic hydrocarbon solvent has passed and causing the extraction solvent to pass through the trapping layer; and
a step 5 of obtaining the extraction solvent having passed through the trapping layer,
wherein the trapping layer includes a trapping material having a carrier containing aluminum oxide and transition metal held on a surface of the carrier.

2. The method for extracting the organic halogen compound according to claim 1, wherein
the carrier is in a form of a grain.

3. The method for extracting the organic halogen compound according to claim 1, wherein
the transition metal is held in a form of fine particles on the surface of the carrier.

4. The method for extracting the organic halogen compound according to claim 1, wherein
the transition metal is at least one selected from a group consisting of silver, copper, and nickel.

5. The method for extracting the organic halogen compound according to claim 1, further comprising:
a step 6 of supplying the aliphatic hydrocarbon solvent having passed through the adsorbent layer in the step 3 to a precedent trapping layer containing a carbon-based material or active magnesium silicate and causing the aliphatic hydrocarbon solvent to pass through the precedent trapping layer, supplying the aliphatic hydrocarbon solvent to the trapping layer and causing the aliphatic hydrocarbon solvent to pass through the trapping layer, and supplying the solvent for extraction of the organic halogen compound to the precedent trapping layer through which the aliphatic hydrocarbon solvent has passed and causing the extraction solvent to pass through the precedent trapping layer; and
a step 7 of obtaining the extraction solvent having passed through the precedent trapping layer.

6. The method for extracting the organic halogen compound according to claim 1, wherein
the solution is a solution containing an organic halogen compound extracted using a solvent from a collector having collected a material layer on a bottom in a hydrosphere or a land surface, food, a biological sample, environmental water, drainage water, electric insulating oil, incinerated ash, or a substance contained in gas.

7. A column for extracting an organic halogen compound from a solution containing the organic halogen compound and an impurity, comprising:
a first column filled with an adsorbent layer capable of treating the impurity; and
a second column detachably coupled to the first column and filled with a trapping layer capable of trapping the organic halogen compound,
wherein the trapping layer includes a trapping material having a carrier containing aluminum oxide and transition metal held on a surface of the carrier.

8. The column for extracting the organic halogen compound according to claim 7, wherein
the second column is further filled with a precedent trapping layer arranged between the adsorbent layer and the trapping layer and containing a carbon-based material or active magnesium silicate.

9. A column for trapping an organic halogen compound contained in an aliphatic hydrocarbon solvent, comprising:
a trapping layer including a trapping material having a carrier containing aluminum oxide and transition metal held on a surface of the carrier.
